(19) United States
(12) Patent Application Publication
(10) Pub. No.: US 2006/0040915 A1
(43) Pub. Date: Feb. 23, 2006

(54) Process for the preparation of cefdinir

(76) Inventors: Yatendra Kumar, Gurgaon (IN); Mohan Prasad, Gurgaon (IN); Ashok Prasad, New Delhi (IN); Shailendra Kumar Singh, Gurgano (IN); Neela Praveen Kumar, Hyderbad (IN)

Correspondence Address: RANBAXY INC., 600 COLLEGE ROAD EAST, SUITE 2100, PRINCETON, NJ, 08540, US

(21) Appl. No.: 10/513,004

(22) PCT Filed: Apr. 26, 2002
(86) PCT No.: PCT/IB02/01410

§ 371 Date: Jul. 14, 2005

(51) Int. Cl. A61K 31/545; C07D 501/14

(52) U.S. Cl. 514/202; 540/222

(57) ABSTRACT

The present invention relates to a process for the preparation of cefdinir on an industrial scale.

### FIELD OF THE INVENTION

The present invention relates to an improved process for the preparation of cefdinir on an industrial scale.

### BACKGROUND OF THE INVENTION

Cefdinir is chemically known as 7-[2-(2-aminothiazol-4-yl)-2-hydroxyiminoacetamido]-3-vinyl-3-cephem-4-carboxylic acid (syn isomer) of 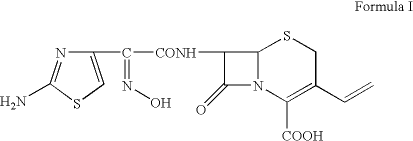
Formula I and was described for the first time in U.S. Pat. No. 4,559,334. It is the third generation cephalosporin antibiotic for oral administration and has a broader antibacterial spectrum than other orally administrable antibiotics. Cefdinir is particularly effective against staphylococci and streptococci.

Several processes have been reported for the preparation of cefdinir. U.S. Pat. No. 4,559,334 describes a process for preparing cefdinir comprising coupling 7-amino-3-vinyl-3-cephem-4-carboxylic acid ester (7-AVCA ester) of Formula P(i), 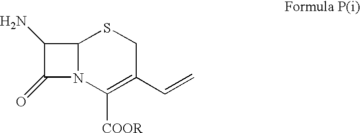
with a reactive derivative of an open chain carboxylic acid of Formula P(ii), 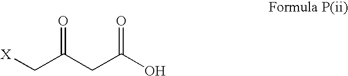
and the resultant 7-amido compound is treated with a nitrosating agent to give an N-oxime compound of Formula P(iii), 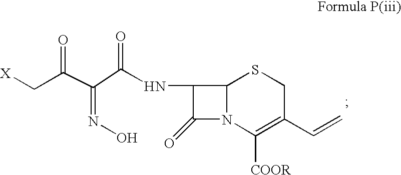
which is then cyclized with thiourea and the carboxyl protecting group is removed to obtain cefdinir. The process is expensive as it involves a number of steps using costly starting compound 7-AVCA.

Japanese patent application 2/790 describes a method involving reacting silyated 7-AVCA with acyloxyiminoacetylhalides followed by removal of acyl group from the condensed product to obtain cefdinir. However, the process requires rigorous anhydrous conditions for the condensation step. Moreover, the preparation of starting compound, requires several synthetic steps and includes the use of phosphorous pentoxide thus making the process unsuitable for production at an industrial scale.

Japanese patent application JP 4/173781 uses formyl protected carboxylic acid which is converted in situ to the acid chloride with phosphorous oxychloride and then coupled with carboxyl protected 7-AVCA of Formula P(i), wherein R is a carboxyl protecting group. The coupled product gives cefdinir in only 22% yield after two successive deprotection steps for removing the formyl group and the carboxyl protecting group, respectively. The use of phosphorous oxychloride is hazardous and highly undesirable at a commercial scale and the low yields due to a large number of steps make the process commercially unattractive.

WO 92/7840 and Japanese patent application JP1/238587 also describes similar processes for preparing cefdinir wherein a carboxyl protected 7-AVCA of Formula P(i) is coupled with an activated ester of 2-aminothiazolyl hydroxyiminoacetamidocarboxylic acid, the amino or the hydroxy group of which are suitably protected. The processes are uneconomical due to several protection and deprotection steps thereby resulting in low overall yields.

WO 01/79211 describes a process for preparing cefdinir, wherein the protecting groups at the carboxyl, hydroxyimino, and amino positions are removed by a mixture of an organic protonic acid and a perhalogenic acid. The use of perhalogenic acid at large scale is undesirable.

U.S. Pat. No. 6,093,814 discloses a process for preparing cefdinir wherein reactive ester of Formula P(iv), 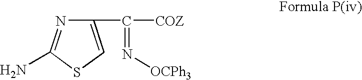
wherein Z is the acid activating group and Ph represents phenyl, is coupled with 7-AVCA of Formula P (v) 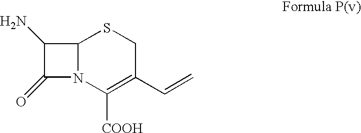
in the presence of N, N-dimethylacetamide (DMAC), and the coupled product is isolated in high yield as p-toluene sulfonic acid addition salt of a DMAC solvate of trityl cefdinir of Formula P(vi), 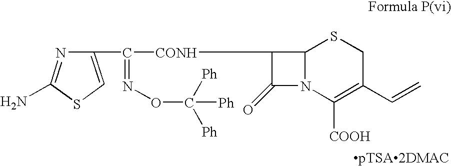
which is treated with an acid to give cefdinir.

Isolation of the compound of formula P(vi) requires addition of large volumes of anti-solvent. Cefdinir obtained following the teachings of U.S. Pat. No. 6,093,814 has a low assay of 90-91% while showing a qualitative purity of 99.1% (by HPLC). This is due to the formation of degradation products and polymerization under the rigorous condition for hydrolysis of compound of Formula P(vi) to cefdinir. Also, the work-up procedure is cumbersome and often requires distilling out the high boiling acids under reduced pressure, which is difficult at large scale.

Therefore, there still exists a need for a simple, efficient and cost effective process for the manufacture of cefdinir of desired purity at a commercial scale. We have now found that trityl cefdinir forms good crystalline DMAC solvated acid addition salts with methanesulfonic acid and sulfuric acid. These salts are easily crystallized out from the reaction mixture without using excess of antisolvents unlike the p-toluene sulfonic acid salt, and their conversion to cefdinir requires very mild conditions yielding pure cefdinir.

### SUMMARY OF THE INVENTION

It is an object of the present invention to provide a process for the preparation of cefdinir of Formula I, 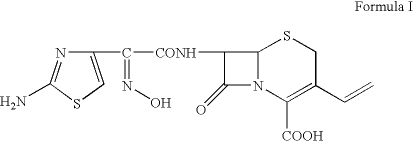
which comprises removing a trityl protecting group in a cefdinir intermediate of Formula II 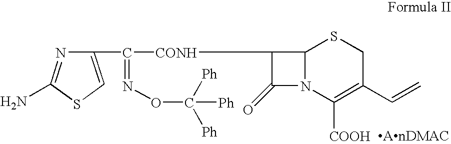
wherein A is sulfuric acid or methanesulfonic acid, n=2 or 3, DMAC is N, N-dimethylacetamide and Ph is phenyl, in the presence or absence of an acid. The cefdinir intermediate compound of Formula II are crystalline compounds and are in the form of a complex with a salt and a solvent. These can be prepared by reacting a reactive ester having the following Formula P(iv), 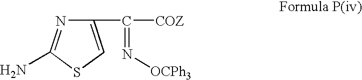
in which Ph represents phenyl, Z represents 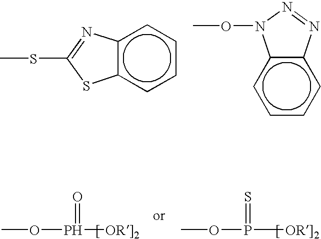
wherein R′ represents C₁-C₄ alkyl or phenyl or R′ together with phosphorus and oxygen atoms to which R′ is attached can form a 5 to 6-membered heterocycle, which is reacted with a 3-cephem derivative having the following Formula P(v), 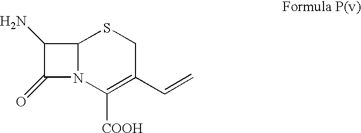
in a solvent comprising N, N-dimethylacetamide (DMAC) in the presence or absence of a base, cooling the reaction mixture to about −10 to 0° C. and then adding sulfuric acid/methanesulfonic acid slowly, maintaining the temperature below 0° C. An antisolvent is then added, the temperature of the mixture is raised to about 30 to 45° C. and the mixture is stirred at the same temperature to crystallize out the compounds of Formula II in good yield and purity.

The reactive ester compound of formula P(iv) and the 3-cephem derivative of formula P(v) are known compounds and can be prepared according to the processes disclosed in European Patent laid-open Publication No. 555,769 and U.S. Pat. No. 4,423,213, which are hereby incorporated herein by reference.

The process for preparing the compound of Formula (II) can be carried out in the presence of a base. Tertiary amines such as triethylamine, tri-n-butylamine, diisopropylethylamine, pyridine, N, N-dimethylaniline, etc. may be used as the base. Preferably, triethylamine or tri-n-butylamine is used.

The antisolvent that is added to crystallize out the compounds of Formula II may be selected from hydrocarbons such as toluene, hexane and lower alkyl ethers such as diethyl ether, diisopropyl ether, or mixture(s) thereof.

Appropriate amounts of antisolvents may be added to crystallize out said compounds. One to two times by volume of the antisolvent (with respect to volume of the reaction solvent used) is usually sufficient to obtain the crystalline compounds in desired yield and purity.

The compounds of Formula II may be converted to cefdinir by conventional methods for removal of the trityl group i.e. acid hydrolysis. However, an important characteristic of the compound of the present invention is that the removal of the trityl group requires very mild conditions. The p-toluene sulfonic acid addition salt provided by U.S. Pat. No. 6,093,814 does not undergo complete hydrolysis without addition of an acid. However, the conversion of compounds of Formula II to cefdinir may be easily achieved either without use of any acid under reflux temperature, or with an acid at ambient temperature.

Conversion of the compound of Formula II to cefdinir is performed in a suitable solvent. Suitable solvents include any solvent, which is inert under the reaction conditions and may be selected from the solvents such as dichloromethane, ethylacetate, toluene, acetonitile, tetrahydrofuran, methanol, isopropanol, water and mixture(s) thereof.

Suitable acid for the conversion include an inorganic acid such as hydrochloric acid, hydrobromic acid, hydroiodic acid, sulfuric acid, etc; a lewis acid such as boron trifluoride, ferrous chloride, stannous chloride, zinc chloride, etc., an organic acid such as acetic acid, formic acid, trifluoroacetic acid, methanesulfonic acid, benzenesulfonic acid, p-toluenesulfonic acid, etc; or an acidic hydrogen ion exchange resin.

Cefdinir obtained by the process of the present invention has a purity greater than 99% and assay greater than 97%. The mild conditions employed for hydrolysis prevent degradation and polymerization of the product.

### DETAILED DESCRIPTION OF THE INVENTION

In the following section preferred embodiments are described by way of examples to illustrate the process of the invention. However, these are not intended in any way to limit the scope of the present invention. Several variants of these examples would be evident to persons ordinarily skilled in the art.

### EXAMPLE 1

### 7β-[2-(2-aminothiazol-4-yl)-2(Z)-trityloximino)acetamido]-3-vinyl-3-cephem-4-carboxylic acid, sulfuric acid salt, 3 N, N-dimethylacetamide solvate

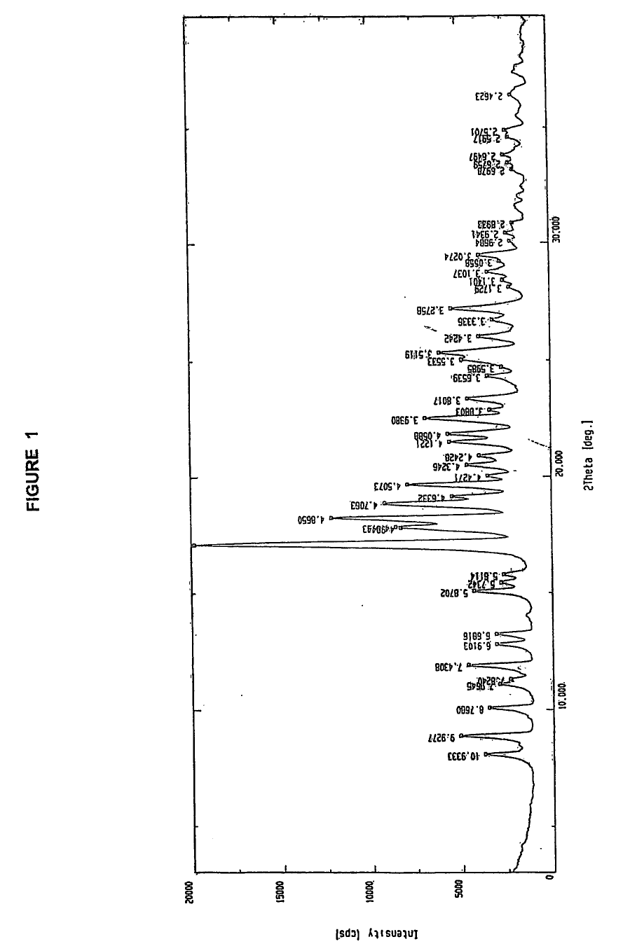

7-amino-3-vinyl-3-cephem-4-carboxylic acid (10 g) was added to N, N-dimethylacetamide (100 ml) followed by the addition of 2-benzothiazolyl (Z)-2-(2-aminothiazol-4-yl)-2-trityloxyiminothioacetate (28.2 g). The reaction mixture was cooled to 10-15° C. and tri-n-butylamine (17.2 g) was added in 20-30 minutes at 10-15° C. The reaction mixture was stirred at ambient temperature for 6-7 hours for completion of reaction. Thereafter, it was cooled to −10° C. and sulfuric acid (13.4 g) was added dropwise in 30 minutes below 0° C. Toluene (100 ml) was added to the reaction mixture under cooled condition followed by the addition of hexane (100 ml). Temperature of the reaction mixture was raised to 35-40° C. for crystallization to take place. The temperature was maintained at 35-40° C. for 30 minutes. The precipitate thus obtained was filtered and washed with toluene and then dried to obtain 41.9 g (yield: 95%) of the title compound as cream colored crystals.
HPLC purity: 98.7%, m.p.=132-135° C.,
Sulfate content (chemical method)=9.86% (w/w),
N, N-Dimethylacetamide content (GC)=25.2% (w/w)
IR (KBr, Cm⁻¹)=3064, 1778, 1688, 1626, 1358, 1195
¹H-NMR (300 MHz, DMSO-d₆) δ: 1.95 (9H, s), 2.76 (9H, s), 2.9 (9H, s), 3.6-3.9 (2H, dd ), 5.2-5.3 (2H, m), 5.5-5.6 (1H, d), 6.7 (1 H, s), 6.9 (1H, m), 7.1-7.3 (17H, m), 10.02-10.05 (1H, d)
FIG. 1 shows the x-ray powder diffraction pattern of a sample prepared according to Example 1.

### EXAMPLE2

### 7β-[2-(2-aminothiazol-4-yl)-2(Z)-(trityoloxyimino)acetamido]-3-vinyl-3-cephem-4-carboxylic acid, methanesulfonic acid salt, 3 N, N-dimethylacetamide solvate

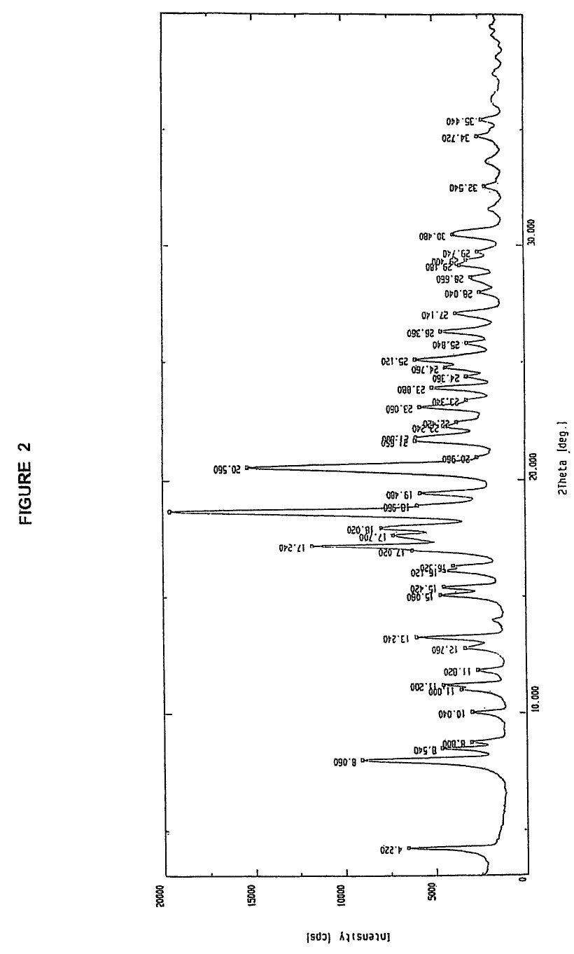

7-amino-3-vinyl-3-cephem-4-carboxylic acid (10 g) was added to N, N-dimethylacetamide (150 ml) followed by the addition of 2-benzothiazolyl (Z)-2-(2-aminothiazol-4-yl)-2-trityloxyiminothioacetate (26.8 g). Tri-n-butylamine (16.78 g) was added to the reaction mixture at 10-15° C. The reaction mixture was stirred at room temperature for 7-8 hours for completion of reaction. Anhydrous methanesulfonic acid (13 g) was added to the reaction mass below 10° C. 15-20 min followed by the addition of diisopropyl ether (150 ml). The reaction mixture was warmed to 30-35° C. for crystallization to take place. The precipitate thus obtained was filtered and washed with diisopropyl ether and then dried to obtain 38.5 g (yield: 96%) of the title compound as off-white crystals.
HPLC purity: 99.3%, m.p.=125-127° C., N, N-Dimethylacetamide content (by GC)=25% (wlw)
IR (KBr, Cm⁻¹) 3062, 1779, 1689, 1620
¹H-NMR (300 MHz, DMSO-d₆) δ: 1.95 (9H, s), 2.3 (3H, s), 2.7 (9H, s), 2.9 (9H, s), 3.6-3.9 (2H, dd), 5.2-5.3 (2H, m), 5.6 (1H,d), 5.9 (1H, m), 6.8 (1H, s), 6.9 (1H, s), 7.2-7.3 (17H, m), 10.05-10.08 (1H, d)
FIG. 2 shows the x-ray powder diffraction pattern of a sample prepared according to Example 2.

### EXAMPLE 3

### 7β-[2-(2-aminothiazol-4-yl)-2(Z)-(trityoloxyimino)acetamido]-3-vinyl-3-cephem-4-carboxylic acid, methanesulfonic acid salt, 2 N, N-dimethylacetamide solvate

7-amino-3-vinyl-3-cephem-4-carboxylic acid (15 g) was added to N, N-dimethylacetamide (225 ml) followed by the addition of 2-benzothiazolyl (Z)-2-(2-aminothiazol-4-yl)-2-trityloxyiminothioacetate (45 g). Tri-n-butylamine (27 g) was added to the reaction mixture at 10-15° C. The reaction mixture was stirred at 25 to 30° C. for 7-8 hours for completion of reaction. Anhydrous methanesulfonic acid (210 g) was added to the reaction mass below 0° C. in 15-20 min followed by the addition of diisopropyl ether (450 ml). The reaction mixture was warmed to 38-40° C. and stirred for 45 minutes for crystallization to take place. The suspension was then cooled to 25 to 30° C. and further stirred for one hour. The precipitate thus obtained was filtered, washed with diisopropyl ether and then dried to obtain 56.7 g (yield: 94.2%) of the title compound as off-white crystals.
HPLC purity: 96.8%, N, N-Dimethylacetamide content (by GC)=21.2% (w/w)

### EXAMPLE4

### 7β-[2-(2-aminothiazol-4-yl)-2-(Z)-trityloxyiminoacetamido]-3-vinyl-3-cephem-4-carboxylic acid

7β-[2-(2-aminothiazol-4-yl)-2-(Z)-trityloxyiminoacetamido]-3-vinyl-3-cephem-4-carboxylic acid, sulfuric acid salt, 3 N, N-dimethylacetamide solvate (25 g) obtained from example 1 was added to methanol (100 ml). The reaction mixture was refluxed for 3.0 hours and thereafter methanol was recovered under reduced pressure. The pH of the concentrated mass was adjusted to 6.5-7.0 by slow addition of saturated solution of sodium bicarbonate. The aqueous layer so obtained was washed with ethylacetate (2×100 ml) followed by the addition of dichloromethane (100 ml). The resultant aqueous layer was degassed and treated with activated carbon under vacuum for 30 minutes, filtered through a cellite and washed with water. The pH of aqueous layer was adjusted to 2.4-2.8 with 6N hydrochloric acid to precipitate cefdinir at its isoelectric point. The crystals thus obtained were stirred at 25-30° C. for 2.0 hours, filtered and washed with water and dried to obtain 9.31 g of title compound as a cream coloured solid (yield: 94%).
HPLC purity: 99.57%
IR (KBr, Cm⁻¹)=3295, 3059, 1767, 1683, 1622, 1352, 1174
¹H-NMR (300 MHz, DMSO-d₆) δ: 3.4-3.8 (2H, m), 5.18 (1H, d), 5.2-5.5 (2H, dd), 5.7 (1H, d), 6.6 (1H, s), 6.8 (m, 1H), 7.1 (2H, brs), 9.48 (1H, d), 11.34 (1H, s).

### EXAMPLE 5

### 7β-[2-(2-aminothiazol-4-yl)-2-(Z)-trityloxyiminoacetamido]-3-vinyl-3-cephem-4-carboxylic acid

7β-[2-(2-aminothiazol-4-yl)-2-(Z)-trityloxyiminoacetamido]-3-vinyl-3-cephem-4-carboxylic acid, sulfuric acid salt, 3 N-N-dimethylacetamide solvate (25 g) was added to dichloromethane (75 ml) and followed by the addition of formic acid (5 ml, 98-100%) to get a clear solution. The reaction mixture was then stirred at room temperature for 3 hours. The reaction mixture was poured into a saturated solution of sodium bicarbonate (150 ml) and pH was adjusted to 6.5-7.0. The resultant layer was separated and aqueous layer was washed with dichloromethane (100 ml), followed by degassing and treatment with activated carbon under vacuum for 30 minutes. The solution was then filtered through a cellite and washed with water. The pH of the clear aqueous layer was adjusted to 2.4-2.8 with 6 N hydrochloric acid to precipitate cefdinir at its isoelectric point. The crystals thus obtained were stirred at 25-30° C. for 2.0 hours, filtered, washed with water and dried to obtain 9.2 g of off white solid (yield: 92.8%).
HPLC purity: 99.7%, IR (KBr, cm⁻¹)=3295, 3059, 1767, 1683, 1622, 1352, 1174
¹H-NMR (300 MHz, DMSO-d₆) δ: 3.4-3.8 (2H, m), 5.18 (1H, d) 5.2-5.5 (2H, dd), 5.7 (1H, m), 6.6(1H, s), 6.8 (1H, m), 7.1 (2H, brs), 9.48 (1H, d), 11.34 (1H, s)

### EXAMPLE 6

### 7β-[2-(2-aminothiazol-4-yl)-2-(Z)-trityloxyiminoacetamido]-3-vinyl-3-cephem-4-carboxylic acid

To the suspension of 7β-[2-(2-aminothiazol-4-yl)-2(Z)-(triyoloxyimino)acetamido]-3-vinyl-3-cephem-4-carboxylic acid, methanesulfonic acid salt, 3N, N-dimethylacetamide solvate (100 g) in dichloromethane (300 ml) was added formic acid (30 ml, 98-100%) and hydrochloric acid (10 ml, 36%) at 10-15° C. The temperature of the mixture was raised to 20-25° C. and stirred for 6-7 hours. The reaction mixture was then poured into a suspension of sodium bicarbonate (85 g) and water (600 ml). The dichloromethane layer was then separated and the aqueous layer was washed with dichloromethane (300 ml). The pH was adjusted to 5.0 with hydrochloric acid and treated with activated carbon. The aqueous layer was acidified to pH 2.5-3.0 with 4N hydrochloric acid. The resulting precipitate was collected by filtration and dried to afford 29.0 g of cefdinir (yield: 73%).
HPLC purity: 99.48%.
Assay (by HPLC): 97.4%

While the present invention has been described in terms of its specific embodiments, certain modifications and equivalents will be apparent to those skilled in the art and are intended to be included within the scope of the present invention.

We claim:

1. A process for the preparation of cefdinir of Formula I, 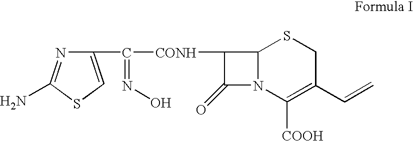
which comprises removing a trityl protecting group in a cefdinir intermediate of formula II, 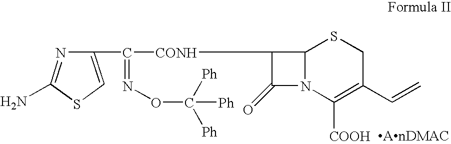
wherein A is sulfuric acid or methanesulfonic acid, n=2 or 3, DMAC is N, N-dimethylacetamide and Ph is phenyl, in the presence or absence of an acid.

2. The process according to claim 1 wherein the compound of Formula II is heated under reflux temperature without an acid to give cefdinir of Formula I.

3. The process according to claim 1 wherein the compound of Formula II is reacted with an acid to give cefdinir of Formula I.

4. The process according to claim 1 wherein the reaction is carried out in the presence of a suitable solvent.

5. The process according to claim 4 wherein the suitable solvent is selected from the group consisting of dichloromethane, ethylacetate, toluene, acetonitile, tetrahydrofuran, methanol, isopropanol, and water.

6. The process according to claim 3 wherein the acid is an inorganic acid, a lewis acid, an organic acid, or an acidic hydrogen ion exchange resin.

7. The process according to claim 6 wherein the inorganic acid is selected from the group consisting of hydrochloric acid, hydrobromic acid, hydroiodic acid, and sulfuric acid.

8. The process according to claim 6 wherein the lewis acid is selected from the group consisting of boron trifluoride, ferrous chloride, stannous chloride, and zinc chloride.

9. The process according to claim 6 wherein the organic acid is selected from the group consisting of acetic acid, formic acid, trifluoroacetic acid, methanesulfonic acid, benzenesulfonic acid, and p-toluenesulfonic acid.

10. A process for the preparation of compound of Formula II 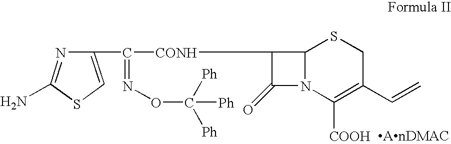
wherein A is sulfuric acid or methanesulfonic acid, n=2 or 3, DMAC is N, N-dimethylacetamide and Ph is phenyl, which comprises reacting a reactive ester having the following formula P(iv), 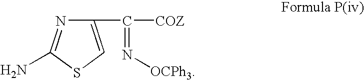
in which Ph represents phenyl, Z represents 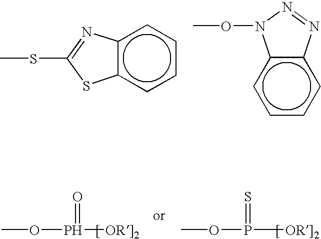
wherein R′ represents C₁-C₄ alkyl or phenyl, with a 3-cephem derivative having the following formula P(v), 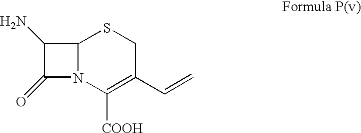
in a solvent comprising N, N-dimethylacetamide (DMAC) in the presence or absence of a base, and then adding sulfuric acid/methane sulfonic acid under cooling at about −10 to 0° C.

11. The process according to claim 10 wherein an antisolvent is added to precipitate the compound of Formula II.

12. The process according to claim 11 wherein the mixture is stirred at a temperature of about 30 to 45° C. after the addition of antisolvent.

13. The process according to claim 11 wherein the antisolvent is selected from the group consisting of hydrocarbons such as toluene, hexane and lower alkyl ethers such as diethyl ether, diisopropyl ether, or mixture(s) thereof.

14. The process of claim 11 wherein an antisolvent is added in an amount which is one to two times by volume with respect to the volume of the reaction solvent.

15. The process according to claim 10 wherein a tertiary amine is used as the base.

16. The process according to claim 15 wherein the tertiary amine is triethylamine or tri-n-butylamine.

17. A crystalline cefdinir intermediate having the following formula II: 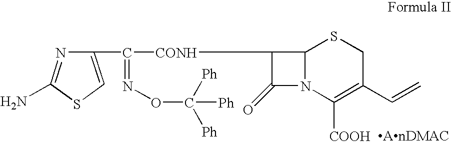
wherein A is sulfuric acid or methanesulfonic acid, n=2 or 3, DMAC is N, N-dimethylacetamide and Ph is phenyl.
